(19) United States
(12) Patent Application Publication
(10) Pub. No.: US 2023/0347009 A1
(43) Pub. Date: Nov. 2, 2023

(54) DISPOSABLE CONTACT LENS CLEANSING DEVICE

(71) Applicant: Val Knight, Dover, DE (US)

(72) Inventor: Val Knight, Dover, DE (US)

(21) Appl. No.: 18/140,906

(22) Filed: Apr. 28, 2023

Related U.S. Application Data
(60) Provisional application No. 63/335,781, filed on Apr. 28, 2022.

(51) Int. Cl. A61L 12/08; A61L 12/14; A61L 12/12

(52) U.S. Cl. CPC A61L 12/086; A61L 12/14; A61L 12/12

(57) ABSTRACT

Described herein is a single-use cleansing device for cleaning a contact lens, and a cleansing solution. The single-use cleansing device includes a first compartment and a second compartment, wherein the first compartment comprises a wipe, and the second compartment comprises a cleansing solution comprising Euphrasia.

### CROSS REFERENCE TO RELATED APPLICATION(S)

The present application claims priority to U.S. Provisional Patent Application No. 63/335,781 filed on Apr. 28, 2022. The entire contents of which are incorporated herein in its entirety.

### FIELD OF THE INVENTION

The present invention relates to a method for cleaning contact lens in a disposable manner using a cleaning solution. Specifically, the method includes a dual compartment product in combination with a cleaning solution containing Euphrasia.

### BACKGROUND OF THE INVENTION

Often, many contact lens wearers experience discomfort, such as dryness or other irritation, while wearing contacts throughout the day. This may be caused by allergies of the wearer, or other irritants. Currently, there are no products to help alleviate any discomfort with a contact by a user while a person is not home. Typically, a user needs clean hands and a rinse to clean the contact lenses, making it difficult to clean a contact lens while a user is not home or supplies are unavailable. Thus, there is a need for a product that could be used to clean or treat contact lens when a wearer is out and experiences discomfort.

### SUMMARY OF THE INVENTION

In some embodiments, a single-use cleansing device may include a first compartment and a second compartment, wherein the first compartment may include a wipe, and the second compartment may include a cleansing solution comprising Euphrasia.

In some embodiments, the wipe may include a moistening agent. In some embodiments, the moistening agent may be isopropyl alcohol.

In some embodiments, the cleansing solution may further include a saline solution. In some embodiments, the saline solution may include sodium chloride. In some embodiments, the sodium chloride is included in the saline solution in an amount of 0.5% to about 1.5%, based on concentration of saline solution.

In some embodiments, the cleansing solution may further include at least one of boric acid, sodium chloride, or sodium borate.

In some embodiments, the cleansing solution may further include formic acid.

In some embodiments, the cleansing solution may further include acetonitrile.

In some embodiments, the cleansing solution may further include water.

In some embodiments, the Euphrasia may be included in an amount of 1.0 mg/mL in the cleansing solution.

In some embodiments, a method is provided. The method includes cleansing a contact lens. The method may include opening a first compartment including a wipe, cleansing the hands of a user using the wipe, opening a second compartment including a cleansing solution comprising Euphrasia, and placing the contact lens into the second compartment.

In some embodiments, the contact lens may sit in the cleansing solution for about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 1 minute.

In some embodiments, the method may further include removing the contact lens from the second compartment.

In some embodiments, any particulates may be removed from the contact lens while in the second compartment.

In some embodiments, a cleansing solution may be provided. The cleansing solution may include Euphrasia and a saline solution. In some embodiments, the cleansing solution may further include at least one of boric acid, sodium chloride, or sodium borate. In some embodiments, the cleansing solution may further include formic acid. In some embodiments, the cleansing solution may further include acetonitrile. In some embodiments, the cleansing solution may further include water. In some embodiments, the cleansing solution may further include Euphrasia is in an amount of 1.0 mg/mL.

### DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates an embodiment of a single-use cleansing device according to the present disclosure; and

FIG. 2 illustrates another embodiment of a single-use cleansing device according to the present disclosure.

### DETAILED DESCRIPTION OF THE INVENTION

The present disclosure relates to a product that employs a simple “dipping, rubbing and cleansing” method of cleaning a contact lens in a convenient, quick and easily disposable manner. The product includes a dual compartment, with one compartment used to dip, rub and cleanse a contact lens and the other compartment is used to store a single use utility to sanitize the hands before touching the contact lens. The product of the present disclosure can be taken on remote locations for use without the otherwise necessary bulk of a large size container because of the small size of the product. The product is designed to be able to carry in pockets or handbags during travel with anticipation of need.

The present disclosure also relates to a cleansing solution. The cleansing solution includes a combination of saline solution, Euphrasia (Eyebright), and/or CBD. The cleansing solution is able to rinse a contact lens while also nourishing the eyes.

The inventor of the present disclosure has found that to address some of the issues described herein, a cleansing device includes a lens cleaning solution in one compartment with nourishing properties for the contact lens, while the other compartment includes a hand cleaning utility allowing for sanitary use. The cleansing device of the present disclosure may be a disposable pouch. The disposable pouch may be made from an eco-friendly film to protect the contents from contaminants.

The cleansing device described herein includes a first compartment and a second compartment. The first compartment houses the nourishing cleansing solution for the contact lens rinse, while the second compartment houses the utility to sanitize the hands. The utility may include a wipe. The wipe may be a disinfectant wipe. The wipe may be a premoistened wipe including alcohol. The alcohol may be isopropyl alcohol.

As used herein, “about” refers to any values that are within a variation of ±10%, such that “about 10” would include from 9 to 11. As used herein, “a,” “an,” or “the” refers to one or more, unless otherwise specified. Thus, for example, reference to “a component” includes a single component as well as a mixture of two or more different components, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

According to an embodiment, a single-use cleansing device is provided. The cleansing device includes two-separate, sealed compartments. The compartments may be connected at their distal ends with a fold-seal arrangement so that the compartments may be collapsed or folded in a billfold fashion. Each compartment is sealed to prevent the egress of dirt or moisture, or to maintain the stability of the cleansing solution.

The single-use cleansing device may include a first compartment and a second compartment, wherein the first compartment may include a wipe, and the second compartment may include a cleansing solution comprising Euphrasia. The wipe may be a pre-moistened wipe or may be a dry, absorbent wipe. In some embodiments, a pre-moistened wipe may include a moistening agent, such as isopropyl alcohol. Isopropyl alcohol is known for its cleaning and germ-killing ability, thus making it suitable for use to clean a user's hands when handling contact lens. Additionally, isopropyl alcohol is not harmful to humans when used externally and dries in a few seconds, such that it does not effect the user when handling contacts near the eye area. It should be understood that other similar non-toxic moistening agents may be used if desired.

In some embodiments, the wipe may be dimensioned in a convenient 6×6 inch size, folded to 2×2 inches. The wipes may be fabricated from any soft, pliable material that is used in the industry. For simplicity, the cleansing device will be described as including a single wipe in the compartment. However, it should be understood that compartment may be modified to hold multiple wipes. In some embodiments, the cleansing device and/or the wipe may be fabricated from biodegradable material, such that the device and wipe may be safely disposed.

The cleansing device described herein is compact and its folded state can easily be accommodated in a purse, pocket, vending situations, etc. In its unfolded state, the cleansing device may be accommodated in the billfold of a wallet. The cleansing device may be easily accessed and safely disposed after use. In some embodiments, the cleansing device may be able to stand vertically. In some embodiments, the cleansing device may be able to close after being able through a closing feature. The closing feature may be a zip lock feature.

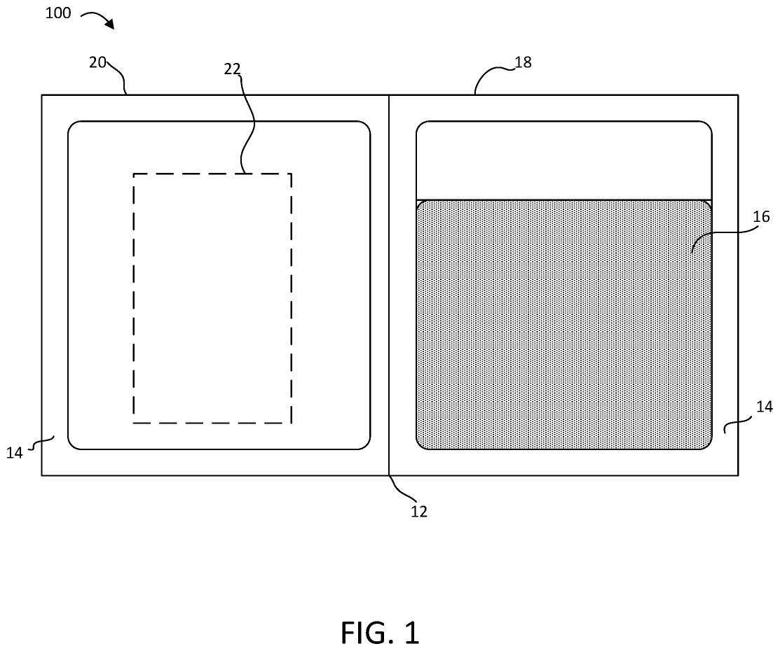

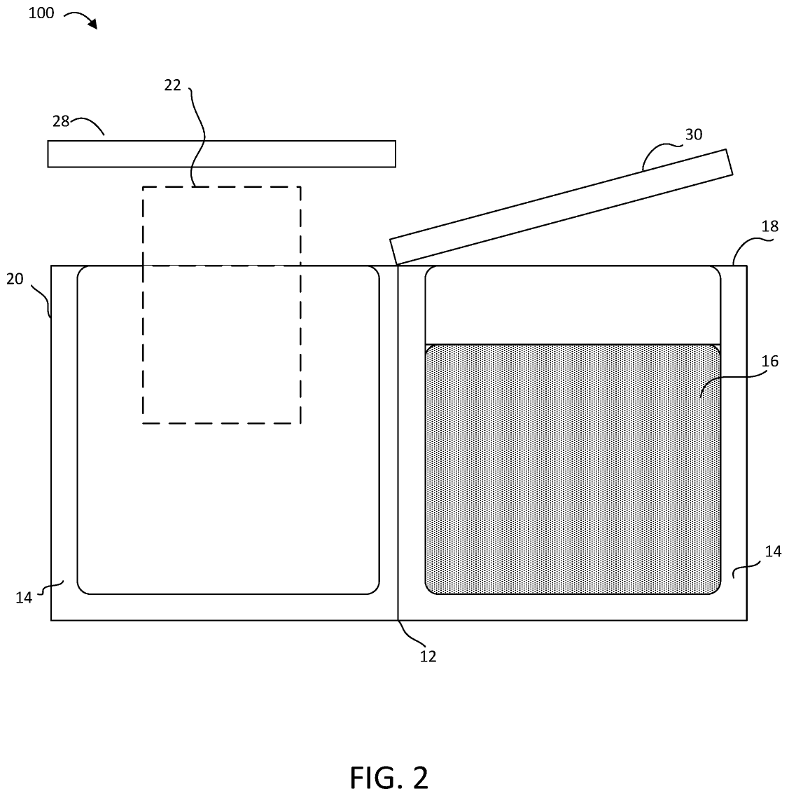

Referring to FIG. 1, a cleansing device 100 is shown. The cleansing device 100 includes a first compartment 20 and a second compartment 18. The first compartment 20 and the second compartment 18 are sealed and connected at their distal ends by a fold member 12. A wipe 22 is housed in the first compartment 20, while the cleansing solution 16 is housed in the second compartment 18. As can also be seen in FIG. 1, the first compartment 20 and the second compartment 18 have a sealed edge 14. It will be understood that a user may tear away at either the first compartment 20 or the second compartment 18 depending on need. In one embodiment, the user will tear away a portion of the first compartment 20 to break the sealed edge 14 and access the wipe 22. The user then may clean their hands before tearing away a portion of the sealed edge 14 of the second compartment 18. This can be seen in FIG. 2.

Referring to FIG. 2, a portion of the first compartment 28 is torn away, to expose the wipe 22. It should be understood that the first compartment may be opened by other means than tearing, such as cutting open. The user may then use the wipe 22 to cleanse his or her hands before handling a contact lens. Further, it should be understood that the wipe 22 does not completely fill the first compartment 20. As can be seen in FIG. 2, a gas 26 may surround the wipe 22 in the compartment. The gas 26 may include air. If the wipe 22 is pre-moistened, then the gas 26 may help maintain the moisture of the wipe 22. A portion of the second compartment 30 is also torn away to access the cleansing solution 16.

The cleansing solution 16 includes a combination of saline solution, and Euphrasia (Eyebright). In some embodiments, the cleansing solution 16 may further include cannabidiol (CBD). In other embodiments, the cleansing solution 16 may include a combination of saline solution and CBD. In some embodiments of the cleansing solution, an aqueous solution including hyaluronic acid, salicylic acid or a salt thereof may be used. The combination of Euphrasia with the saline solution has been found to effectively clean and nourish a contact lens. As understood herein, the term “Euphrasia” refers to a plant extract. The extract can be obtained from plants or parts thereof, for example water. Extracts can also be liquid or solid in nature. In some embodiments, Euphrasia may be obtained from fresh or dried above-parts of Euphrasia officinalis, such as leaves, flowers and/or stems. Extracts of the entire plant of Euphrasia officinalis may be used. Euphrasia is also known to one of skill in the art to as Eyebright and also refers to all type of the Euphrasia genus normally indicated under this term, for example, Euphrasia stricta, Euphrasia montana and/or Euphraisa rostkoviana.

In some embodiments, the cleansing solution may further include at least one of boric acid, sodium chloride, or sodium borate. In some embodiments, the cleansing solution may further include formic acid. In some embodiments, the cleansing solution may further include acetonitrile. In some embodiments, the cleansing solution may further include water.

In some embodiments, the Euphrasia may be included in an amount of 1.0 mg/mL in the cleansing solution. In other embodiments, the Euphrasia may be included in an amount of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, or about 2.5 mg/mL. In some embodiments, the Euphrasia may be included in an amount of about 0.1 mg/mL to about 5 mg/mL, about 0.2 mg/mL to about 4.5 mg/mL, about 0.3 mg/mL to about 4.0 mg/mL, about 0.4 mg/mL to about 3.5 mg/mL, about 0.5 mg/mL to about 3.0 mg/mL, about 0.6 mg/mL to about 2.5 mg/mL, about 0.7 mg/mL to about 2.0 mg/mL, about 0.8 mg/mL to about 1.5 mg/mL, or about 0.8 mg/mL to about 1.2 mg/mL.

In some embodiments of the cleansing solution, the cleansing solution may include sodium chloride. The sodium chloride may be included in an amount of about 5 mg/mL to about 20 mg/mL, about 7 mg/mL to about 18 mg/mL, about 9 mg/mL to about 16 mg/mL, or about 11 mg/mL to about 15 mg/mL, based on total formulation. In some embodiments, the sodium chloride may be included in an amount of about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, about 15 mg/mL, about 15.5 mg/mL, about 16 mg/mL, about 16.5 mg/mL, about 17 mg/mL, about 17.5 mg/mL, about 18 mg/mL, about 18.5 mg/mL, about 19 mg/mL, about 19.5 mg/mL, or about 20 mg/mL.

In some embodiments of the cleansing solution, boric acid may be included. The boric acid may be included in an amount of about 1.0 mg/mL to about 10 mg/mL, about 2 mg/mL to about 9 mg/mL, about 3 mg/mL to about 8 mg/mL, or about 4 mg/mL to about 7 mg/mL. In some embodiments, the boric acid may be included in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL.

In some embodiments, sodium borate may be included in the cleansing solution. The sodium borate may be included in an amount of about 0.001 mg/mL to about 1 mg/mL, about 0.01 mg/mL to about 0.9 mg/mL, about 0.1 mg/mL to about 0.8 mg/mL, about 0.2 mg/mL to about 0.7 mg/mL, about 0.3 mg/mL to about 0.6 mg/mL, or about 0.4 mg/mL to about 0.5 mg/mL. In some embodiments, the sodium borate may be included in an amount of about 0.001 mg/mL, about 0.01 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL.

In some embodiments, the cleansing solution may have a pH of about 7.0 to about 8.0, or about 7.1 to about 7.5.

Referring back to FIG. 2, the second compartment 18 housing the cleansing solution 16 may include a textured surface. A textured surface may aid in rinsing a contact lens, when the contact lens is placed in the second compartment 18 for cleaning. The second compartment 18 includes a gap 24 such that when there is space so that a contact lens can be inserted into the second compartment 18.

In some embodiments, a method of cleansing/cleaning is provided. The method includes cleansing a contact lens using the cleansing device described herein. The method may include opening a first compartment including a wipe, where the wipe is used to clean the hands of a user. The user may then open the second compartment including a cleansing solution comprising Euphrasia as described above, where the contact lens is placed into the second compartment.

In some embodiments, the contact lens may sit in the cleansing solution for about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 1 minute. In some embodiments, a user may gently shake the cleansing device to move the contact lens and ensure cleaning.

In some embodiments, the method may further include removing the contact lens from the second compartment.

In some embodiments, any particulates may be removed from the contact lens while in the second compartment.

What is claimed is:

1. A single-use cleansing device comprises a first compartment and a second compartment,
wherein the first compartment comprises a wipe, and the second compartment comprises a cleansing solution comprising Euphrasia.

2. The single-use cleansing device of claim 1, wherein the wipe comprises a moistening agent.

3. The single-use cleansing device of claim 2, wherein the moistening agent comprises isopropyl alcohol.

4. The single-use cleansing device of claim 1, wherein the cleansing solution further comprises a saline solution.

5. The single-use cleansing device of claim 1, wherein the cleansing solution further comprises at least one of boric acid, sodium chloride, or sodium borate.

6. The single-use cleansing device of claim 1, wherein the cleansing solution further comprises formic acid.

7. The single-use cleansing device of claim 1, wherein the cleansing solution further comprises acetonitrile.

8. The single-use cleansing device of claim 1, wherein the cleansing solution further comprises water.

9. The single-use cleansing device of claim 1, wherein the Euphrasia is included in an amount of 1.0 mg/mL.

10. A cleansing solution comprising:
Euphrasia and a saline solution.

11. The cleansing solution of claim 10, wherein the Euphrasia comprises Euphrasia stricta, Euphrasia montana, Euphraisa rostkoviana, or a combination thereof.

12. The cleansing solution of claim 10, wherein the saline solution comprises sodium chloride.

13. The cleansing solution of claim 12, wherein the sodium chloride is included in the saline solution in an amount of 0.5% to about 1.5%, based on concentration of saline solution.

14. The cleansing solution of claim 10, further comprising at least one of boric acid, sodium chloride, or sodium borate.

15. The cleansing solution of claim 10, further comprising formic acid.

16. The cleansing solution of claim 10, further comprising acetonitrile.

17. The cleansing solution of claim 10, further comprising water.

18. The cleansing solution of claim 10, wherein the Euphrasia is included in an amount of 1.0 mg/mL.

19. The cleansing solution of claim 10, further comprising cannabidiol.
